(19) United States
(12) Patent Application Publication
(10) Pub. No.: US 2014/0371304 A1
(43) Pub. Date: Dec. 18, 2014

(54) METHOD FOR SUPPRESSING TUMORIGENICITY OF STEM CELLS

(71) Applicant: SUCAMPO AG, ZUG (CH)

(72) Inventors: Ryuji UENO, St. Michaels, MD (US); Sachiko KUNO, St Michaels, MD (US); John CUPPOLETTI, Cincinnati, OH (US)

(73) Assignee: SUCAMPO AG, ZUG (CH)

(21) Appl. No.: 14/301,911

(22) Filed: Jun. 11, 2014

Related U.S. Application Data
(60) Provisional application No. 61/834,541, filed on Jun. 13, 2013.
(60) Provisional application No. 61/886,257, filed on Oct. 3, 2013.

(51) Int. Cl. C07D 311/94; C07C 69/738

(52) U.S. Cl. CPC C07D 311/94; C07C 69/738
USPC 514/456; 514/530; 549/396; 560/121; 435/375

(57) ABSTRACT

A method for suppressing tumorigenicity of stem cells in a mammalian subject, which comprises administering an effective amount of a fatty acid derivative and a method for suppressing tumorigenicity of stem cells of a mammalian subject, which comprises contacting said stem cell with an effective amount of a fatty acid derivative, are provided.

### CROSS REFERENCE TO RELATED APPLICATIONS

This application claims the benefit of U.S. provisional Patent Application No. 61/834,541 filed on Jun. 13, 2013 and U.S. provisional Patent Application No. 61/886,257 filed on Oct. 3, 2013, the entire contents of which are incorporated by reference herein.

### TECHNICAL FIELD

The present invention relates to a method for suppressing tumorigenicity of stem cells in a mammalian subject with an effective amount of a fatty acid derivative.

### BACKGROUND

Human embryonic stem cells (HESCs) have two unique properties: self-renewal, the ability to proliferate indefinitely while maintaining their cellular identity; and pluripotency, the ability to differentiate into all of the cell types that comprise the embryo proper. These traits make HESCs promising for future regenerative medicine, but the same traits also make them tumorigenic, and consequently hinder the fulfilment of their clinical potential. Thus, HESCs could be aptly described as ‘double-edged swords’, as their defining characteristics make them both powerful and dangerous.

The discovery of human induced pluripotent stem cells (HiPSCs) has revolutionized the field of pluripotent stem cell research for two main reasons: it changed the perception of cellular reprogramming, showing that the plasticity of somatic cells is much greater than had previously been thought; and it offered an appealing solution to the likely immune rejection of HESC-derived cells on their transplantation into an unmatched patient, thus providing new and exciting avenues for patient-specific cell therapy. Importantly, HiPSCs also provide a possible solution to the ethical objections that have been raised against the use of HESCs, which is a highly controversial topic in many countries.

Although constituting a huge leap towards overcoming immunogenic and ethical obstacles, the translation of HiPSCs into the clinic faces the same substantial tumorigenicity problem as that of HESCs. Sharing with HESCs their basic properties of self-renewal and pluripotency, HiPSCs are doomed to share with them the other ‘edge of the sword’ (NATURE REVIEWS|CANCER VOLUME 11|APRIL 2011|268-277).

Fatty acid derivatives are members of class of organic carboxylic acids, which are contained in tissues or organs of human or other mammals, and exhibit a wide range of physiological activity. Some fatty acid derivatives found in nature generally have a prostanoic acid skeleton as shown in the formula (A):

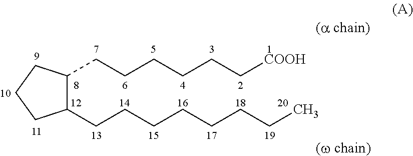 [SMILES: CCCCCCCCC1CCC[C@@H]1CCCCCCC(=O)O]

On the other hand, some of synthetic prostaglandin (PG) analogues have modified skeletons. The primary PGs are classified into PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-, and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGs are known to have various pharmacological and physiological activities, for example, vasodilatation, inducing of inflammation, platelet aggregation, stimulating uterine muscle, stimulating intestinal muscle, anti-ulcer effect and the like.

Prostones, having an oxo group at position 15 of prostanoic acid skeleton (15-keto type) and having a single bond between positions 13 and 14 and an oxo group at position 15 (13,14-dihydro-15-keto type), are fatty acid derivatives known as substances naturally produced by enzymatic actions during metabolism of the primary PGs and have some therapeutic effect.

U.S. Patent publication No. 2009-0209643 to Ueno et al. describes specific prostaglandin compounds are useful for modulating stem cell proliferation and/or differentiation.

### DISCLOSURE OF THE INVENTION

The present invention relates to a method for suppressing tumorigenicity of stem cells in a mammalian subject, which comprises administering to the subject in need thereof an effective amount of a fatty acid derivative represented by the formula (I):

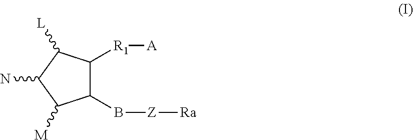

wherein L, M and N are hydrogen, hydroxy, halogen, lower alkyl, hydroxy(lower)alkyl, lower alkanoyloxy or oxo, wherein the five-membered ring may have at least one double bond;

A is —CH₃, or —CH₂OH, —COCH₂OH, —COOH or a functional derivative thereof;

B is single bond, —CH₂—CH₂—, —CH═CH—, —CH₂—CH₂—CH₂—, —CH═CH—CH₂—, —CH₂—CH═CH—, —C≡C—CH₂— or —CH₂—C≡C—;

Z is

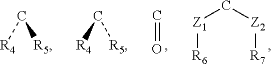

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

The present invention further relates to a method for suppressing tumorigenicity of stem cells of a mammalian subject, which comprises contacting said stem cells with an effective amount of the fatty acid derivative.

The present invention further relates to a supplement or a medium for culturing stem cells such as embryonic stem cells comprising the fatty acid derivative.

### DETAILED DESCRIPTION OF THE INVENTION

The nomenclature of the fatty acid derivative used herein is based on the numbering system of the prostanoic acid represented in the above formula (A).

The formula (A) shows a basic skeleton of the C-20 fatty acid derivative, but the present invention is not limited to those having the same number of carbon atoms. In the formula (A), the numbering of the carbon atoms which constitute the basic skeleton of the fatty acid derivatives starts at the carboxylic acid (numbered 1), and carbon atoms in the α-chain are numbered 2 to 7 towards the five-membered ring, those in the ring are 8 to 12, and those in the α-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of carboxy group (C−1). Similarly, when the number of carbon atoms is decreased in the ω-chain, the number is deleted in the order starting from position 20; and when the number of carbon atoms is increased in the ω-chain, the carbon atoms at the position or later are named as a substituent at position 20. Stereochemistry of the compounds is the same as that of the above formula (A) unless otherwise specified.

In general, each of PGD, PGE and PGF represents a fatty acid derivative having hydroxy groups at positions 9 and/or 11, but in the present specification they also include those having substituents other than the hydroxy groups at positions 9 and/or 11. Such compounds are referred to as 9-deoxy-9-substituted-fatty acid derivatives or 11-deoxy-11-substituted-fatty acid derivatives. A fatty acid derivative having hydrogen in place of the hydroxy group is simply named as 9- or 11-deoxy-fatty acid derivative.

As stated above, the nomenclature of a fatty acid derivative is based on the prostanoic acid skeleton. In the case the compound has similar partial structure as the primary PG, the abbreviation of “PG” may be used. Thus, a fatty acid derivative whose α-chain is extended by two carbon atoms, that is, having 9 carbon atoms in the α-chain is named as 2-decarboxy-2-(2-carboxyethyl)-PG compound. Similarly, a fatty acid derivative having 11 carbon atoms in the α-chain is named as 2-decarboxy-2-(4-darboxybutyl)-PG compound. Further, a fatty acid derivative whose ω-chain is extended by two carbon atoms, that is, having 10 carbon atoms in the ω-chain is named as 20-ethyl-PG compound. These compounds, however, may also be named according to the IUPAC nomenclatures.

Examples of the analogues including substitution compounds or derivatives of the above described fatty acid derivative include a fatty acid derivative whose carboxy group at the end of the alpha chain is esterified; a fatty acid derivative whose α chain is extended, a physiologically acceptable salt thereof, a fatty acid derivative having a double bond between positions 2 and 3 or a triple bond between positions 5 and 6; a fatty acid derivative having substituent(s) on carbon atom(s) at position(s) 3, 5, 6, 16, 17, 18, 19 and/or 20; and a fatty acid derivative having a lower alkyl or a hydroxy (lower) alkyl group at position 9 and/or 11 in place of the hydroxy group.

According to the present invention, preferred substituents on the carbon atom at position(s) 3, 17, 18 and/or 19 include alkyl having 1-4 carbon atoms, especially methyl and ethyl. Preferred substituents on the carbon atom at position 16 include lower alkyls such as methyl and ethyl, hydroxy, halogen atom such as chlorine and fluorine, and aryloxy such as trifluoromethylphenoxy. Preferred substituents on the carbon atom at position 17 include lower alkyl such as methyl and ethyl, hydroxy, halogen atom such as chlorine and fluorine, and aryloxy such as trifluoromethylphenoxy. Preferred substituents on the carbon atom at position 20 include saturated or unsaturated lower alkyl such as C₁₋₄ alkyl, lower alkoxy such as C₁₋₄ alkoxy, and lower alkoxy alkyl such as C₁₋₄ alkoxy-C₁₋₄ alkyl. Preferred substituents on the carbon atom at position 5 include halogen atoms such as chlorine and fluorine. Preferred substituents on the carbon atom at position 6 include an oxo group forming a carbonyl group. Stereochemistry of PGs having hydroxy, lower alkyl or hydroxy(lower)alkyl substituent on the carbon atom at positions 9 and 11 may be α, β or a mixture thereof.

Further, the above described analogues or derivatives may have a ω chain shorter than that of the primary PGs and a substituent such as alkoxy, cycloalkyl, cycloalkyloxy, phenoxy and phenyl at the end of the truncated ω-chain.

A fatty acid derivative used in the present invention is represented by the formula (I):

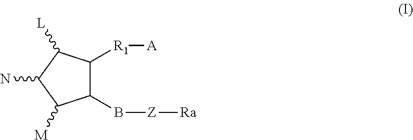

wherein L, M and N are hydrogen, hydroxy, halogen, lower alkyl, hydroxy(lower)alkyl, lower alkanoyloxy or oxo, wherein the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

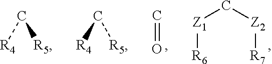

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

A preferred compound used in the present invention is represented by the formula (II):

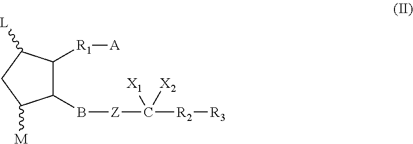

wherein L and M are hydrogen atom; hydroxy, halogen, lower alkyl, hydroxy(lower)alkyl, lower alkanoyloxy or oxo, wherein the five-membered ring may have one or more double bonds;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

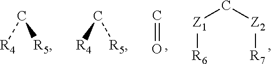

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;

X₁ and X₂ are hydrogen, lower alkyl, or halogen;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur;

R₂ is a single bond or lower alkylene; and

R₃ is lower alkyl, lower alkoxy, lower alkanoyloxy, cyclo(lower)alkyl, cyclo(lower)alkyloxy, aryl, aryloxy, heterocyclic group or heterocyclic-oxy group, and at least one of carbon atom in the aliphatic hydrocarbon is optionally substituted by oxygen, nitrogen or sulfur.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydrogen, hydroxy and oxo, and especially, L and M are both hydroxy, or L is oxo and M is hydrogen or hydroxy.

Preferred example of A is —COOH, its pharmaceutically acceptable salt, ester or amide thereof.

Preferred example of X₁ and X₂ are both being hydrogens or halogen atoms, and in case of halogen atoms, more preferably, fluorine atoms, so called 16,16-difluoro type.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur. Examples of R₁ include, for example, the following groups:

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—O—CH₂—,

—CH₂—CH═CH—CH₂—O—CH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂—,

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—CF—C—CH₂—CH₂—CH₂—CH₂—CH₂—, and

—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains having one carbon atom. Further, at least one carbon atom in the aliphatic hydrocarbon is optionally substituted by oxygen, nitrogen or sulfur.

In embodiments of the present invention, representative compounds of the formula (I) or (II) include compounds of the formula (I) wherein Ra is substituted by halogen and/or Z is C═O;

compounds of the formula (II) wherein one of X₁ and X₂ is substituted by halogen and/or Z is C═O;
compounds of the formula (II) wherein L is ═O or —OH, M is H or OH, A is COOH or a functional derivative thereof, B is —CH₂—CH₂—, Z is C═O, X₁ is halogen (e.g. X₁ is Cl, Br, I or F) or hydrogen, X₂ is halogen (e.g. X₂ is Cl, Br, I or F) or hydrogen, R₁ is a saturated or unsaturated bivalent straight C₆ aliphatic hydrocarbon residue, R₂ is a single bond, and R₃ is straight or branched lower alkyl (e.g. C₄₋₆ alkyl) optionally substituted by oxygen, nitrogen or sulfur;
compounds of the formula (II) wherein L is ═O, M is OH, A is COOH or a functional derivative thereof, B is —CH₂—CH₂—, Z is C═O, X₁ is halogen (e.g. X₁ is Cl, Br, I or F) or hydrogen, X₂ is halogen (e.g. X₂ is Cl, Br, I or F) or hydrogen, R₁ is a saturated or unsaturated bivalent straight C₆ aliphatic hydrocarbon residue, R₂ is a single bond, and R₃ is straight or branched lower alkyl optionally substituted by oxygen, nitrogen or sulfur;
compounds of the formula (II) wherein L is ═O, M is OH, A is COOH or a functional derivative thereof, B is —CH₂—CH₂—, Z is C═O, X₁ and X₂ are halogen atoms (e.g. X₁ and X₂ are Cl, Br, I or F), R₁ is a saturated or unsaturated bivalent straight C₆ aliphatic hydrocarbon residue, R₂ is a single bond, and R₃ is straight or branched lower alkyl (e.g. C₄ alkyl or C₅ alkyl);
compounds of the formula (II) wherein L is ═O, M is OH, A is COOH or a functional derivative thereof, B is —CH₂—CH₂—, Z is C═O, X₁ and X₂ are fluorine atoms, R₁ is a saturated or unsaturated bivalent straight C₆ aliphatic hydrocarbon residue, R₂ is a single bond, and R₃ is straight or branched lower alkyl (e.g. C₄ alkyl or C₅ alkyl);
compounds of the formula (II) wherein L is ═O, M is H or OH, A is COOH or a functional derivative thereof, B is —CH₂—CH₂—, Z is C═O, X₁ and X₂ are halogen atoms (e.g. X₁ and X₂ are Cl, Br, I or F), R₁ is a saturated or unsaturated bivalent straight C₆ aliphatic hydrocarbon residue, R₂ is a single bond, and R₃ is —CH₂—CH₂—CH₂—CH₃ or —CH₂—CH(CH₃)—CH₂—CH₃;

In further embodiment, representative compounds used in the present invention include (−)-7-[(2R,4aR,5R,7aR)-2-(1,1-difluoropentyl)-2-hydroxy-6-oxooctahydrocyclopenta[b]pyran-5-yl]heptanoic acid (lubiprostone), (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid (cobiprostone), (+)-isopropyl (Z)-7-[(1R,2R,3R,5S)-3,5-dihydroxy-2-(3-oxodecyl)cyclopentyl]hept-5-enoate (isopropyl unoprostone) (−)-7-[(1R,2R)-2-(4,4-difluoro-3-oxooctyl)-5-oxocyclopentyl]heptanoic acid, (E)-7-[(1R,2R)-2-(4,4-difluoro-3-oxooctyl)-5-oxocyclopentyl]hept-2-enoic acid, a tautomeric isomer thereof and a functional derivative thereof.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

In the present invention, the fatty acid derivative which is dihydro between 13 and 14, and keto (═O) at 15 position may be in the keto-hemiacetal equilibrium by formation of a hemiacetal between hydroxy at position 11 and keto at position 15.

For example, it has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the present invention includes both isomers.

Further, the fatty acid derivatives used in the invention include the bicyclic compound and analogs or derivatives thereof.

The bicyclic compound is represented by the formula (III)

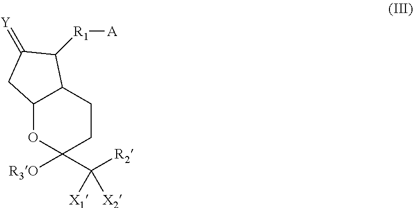

wherein, A is —CH₃, or —CH₂OH, —COCH₂OH, —COOH or a functional derivative thereof;

X₁′ and X₂′ are hydrogen, lower alkyl, or halogen;

Y is

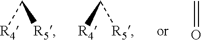

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated divalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

R₃′ is hydrogen, lower alkyl, cyclo(lower)alkyl, aryl or heterocyclic group.

Furthermore, while the compounds used in the invention may be represented by a formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161 and 6,242,485 (these cited references are herein incorporated by reference).

The mammalian subject may be any mammalian subject including a human. The compound may be applied systemically or topically. Usually, the compound may be administered by oral administration, intranasal administration, inhalational administration, intravenous injection (including infusion), subcutaneous injection, ocular topical administration, intra rectal administration, intra vaginal administration, transdermal administration and the like.

The dose may vary depending on the strain of the animal, age, body weight, symptom to be treated, desired therapeutic effect, administration route, term of treatment and the like. A satisfactory effect can be obtained by systemic administration 1-4 times per day or continuous administration at the amount of 0.00001-500 mg/kg per day, more preferably 0.0001-100 mg/kg.

The compound may preferably be formulated in a pharmaceutical composition suitable for administration in a conventional manner. The composition may be those suitable for oral administration, intranasal administration, ocular topical administration, inhalational administration, injection or perfusion as well as it may be an external agent, suppository or pessary.

The composition of the present invention may further contain physiologically acceptable additives. Said additives may include the ingredients used with the present compounds such as excipient, diluent, filler, resolvent, lubricant, adjuvant, binder, disintegrator, coating agent, cupsulating agent, ointment base, suppository base, aerozoling agent, emulsifier, dispersing agent, suspending agent, thickener, tonicity agent, buffering agent, soothing agent, preservative, antioxidant, corrigent, flavor, colorant, a functional material such as cyclodextrin and biodegradable polymer, stabilizer. The additives are well known to the art and may be selected from those described in general reference books of pharmaceutics.

The amount of the above-defined compound in the composition of the invention may vary depending on the formulation of the composition, and may generally be 0.000001-10.0%, more preferably 0.00001-5.0%, most preferably 0.0001-1%.

Examples of solid compositions for oral administration include tablets, troches, sublingual tablets, capsules, pills, powders, granules and the like. The solid composition may be prepared by mixing one or more active ingredients with at least one inactive diluent. The composition may further contain additives other than the inactive diluents, for example, a lubricant, a disintegrator and a stabilizer. Tablets and pills may be coated with an enteric or gastroenteric film, if necessary. They may be covered with two or more layers. They may also be adsorbed to a sustained release material, or microcapsulated. Additionally, the compositions may be capsulated by means of an easily degradable material such gelatin. They may be further dissolved in an appropriate solvent such as fatty acid or its mono, di or triglyceride to be a soft capsule. Sublingual tablet may be used in need of fast-acting property.

Examples of liquid compositions for oral administration include emulsions, solutions, suspensions, syrups and elixirs and the like. Said composition may further contain a conventionally used inactive diluents e.g. purified water or ethyl alcohol. The composition may contain additives other than the inactive diluents such as adjuvant e.g. wetting agents and suspending agents, sweeteners, flavors, fragrance and preservatives.

The composition of the present invention may be in the form of spraying composition, which contains one or more active ingredients and may be prepared according to a known method.

Example of the intranasal preparations may be aqueous or oily solutions, suspensions or emulsions comprising one or more active ingredient. For the administration of an active ingredient by inhalation, the composition of the present invention may be in the form of suspension, solution or emulsion which can provide aerosol or in the form of powder suitable for dry powder inhalation. The composition for inhalational administration may further comprise a conventionally used propellant.

Examples of the injectable compositions of the present invention for parenteral administration include sterile aqueous or non-aqueous solutions, suspensions and emulsions. Diluents for the aqueous solution or suspension may include, for example, distilled water for injection, physiological saline and Ringer's solution.

Non-aqueous diluents for solution and suspension may include, for example, propylene glycol, polyethylene glycol, vegetable oils such as olive oil, alcohols such as ethanol and polysorbate. The composition may further comprise additives such as preservatives, wetting agents, emulsifying agents, dispersing agents and the like. They may be sterilized by filtration through, e.g. a bacteria-retaining filter, compounding with a sterilizer, or by means of gas or radioisotope irradiation sterilization. The injectable composition may also be provided as a sterilized powder composition to be dissolved in a sterilized solvent for injection before use.

The present external agent includes all the external preparations used in the fields of dermatology and otolaryngology, which includes ointment, cream, lotion and spray.

Another form of the present invention is suppository or pessary, which may be prepared by mixing active ingredients into a conventional base such as cacao butter that softens at body temperature, and nonionic surfactants having suitable softening temperatures may be used to improve absorbability.

According to the present invention, the fatty acid derivatives of the present invention are useful for suppressing tumorigenicity of stem cells in a mammalian subject, which comprises administering to the subject in need thereof.

Another embodiment of the present invention includes a method for suppressing tumorigenicity of stem cells of a mammalian subject, which comprises contacting exogenous or endogenous stem cells with an effective amount of the fatty acid derivative.

The method can be used on cells in culture, e.g. in vitro or ex vivo. For example, stem cells or progenitor cells can be cultured in vitro in culture medium and the contacting step can be effected by adding one or more fatty acid derivatives to the culture medium in an amount sufficient, e.g. at the amount of 1×10⁻¹²-1×10⁻³ mol/l. Thus, in one embodiment, one or more fatty acid derivatives provided by the present invention may be used as a supplement or a medium for culturing stem cells (e.g. embryonic stem cells) in vitro. The supplement provided by the present invention may further contain LIF, BMP-4, GDF6 and/or bFGF. The supplement may contain the fatty acid derivative provided by the present invention, LIF, BMP-4, GDF6 and bFGF, respectively, so that when the supplement is added into a culture medium, the content of the fatty acid derivative provided by the present invention, LIF, BMP-4, GDF6 and bFGF in the culture medium is 0.01 nM-100 μM or 0.1 μM-10 μM (regarding the fatty acid) and 0.1 ng/ml-300 ng/ml (regarding LIF, BMP-4, GDF6 and bFGF). For example, in the case where the supplement is 100-fold diluted to be added in a medium for culturing stem cells, the supplement may contain 1 nM-10 mM or 10 μM-1000 μM of the fatty acid derivative provided by the present invention, 0.1 μg/ml-30 μg/ml of LIF, 0.1 μg/ml-30 μg/ml of BMP-4, 0.1 μg/ml-30 μg/ml of GDF6 and/or 0.1 μg/ml-30 μg/ml of bFGF. The supplement may be prepared by dissolving one or more fatty acid derivatives provided by the present invention (e.g. ((−)-7-[(2R,4aR,5R,7aR)-2-(1,1-difluoropentyl)-2-hydroxy-6-oxooctahydrocyclopenta[b]pyran-5-yl]heptanoic acid), ((+)-isopropyl (Z)-7-[(1R,2R,3R,5S)-3,5-dihydroxy-2-(3-oxodecyl)cyclopentyl]hept-5-enoate)) together with LIF and/or BMP-4 in aqueous solution (e.g. saline, PBS etc.).

One or more fatty acid derivatives provided by the present invention may be contained in a medium used for culturing stem cells (e.g. embryonic stem cells). For example, 0.01 nM-100 μM or 0.1 μM-10 μM of fatty acid derivatives provided by the present invention may be contained in a medium used for culturing stem cells.

Any medium which is known to be used for culturing stem cells (e.g. embryonic stem cells) may be used for culturing stem cells, in which one or more fatty acid derivatives provided by the present invention are dissolved. Examples of medium which is known to be used for culturing stem cells include, but are not limited to, ESGRO complete serum free clonal grade medium, Iscove's-modified Dulbecco's medium (IMDM/Ham's F-12), N2B27 medium, DMEM, DMEM-F12 supplemented with NON-ESSENTIAL AMINO ACID, 200 mM L-GLUTMINE, KnockOut Serum Replacement™ (KSR, Invitrogen), KnockOut DMEM (Invitrogen) and KnockOut DM/F12 (Invitrogen). The medium may be further supplemented with LIF, BMP-4, GDF6 and/or bFGF. The content of LIF, BMP-4, GDF6 and bFGF may be 0.1 ng/ml-300 ng/ml, 1 ng/ml-200 ng/ml, 10 ng/ml or 200 ng/ml, respectively. A medium used for culturing stem cells containing the fatty acid derivatives provided by the present invention may be prepared by dissolving the fatty acid derivatives provided by the present invention in a medium which is known to be used for culturing stem cells. For example, a medium used for culturing stem cells containing 0.01 nM-100 μM of the fatty acid derivatives provided by the present invention may be prepared by dissolving (−)-7-[(2R,4aR,5R,7aR)-2-(1,1-difluoropentyl)-2-hydroxy-6-oxooctahydrocyclopenta[b]pyran-5-yl]heptanoic acid or (+)-isopropyl (Z)-7-[(1R,2R,3R,5S)-3,5-dihydroxy-2-(3-oxodecyl)cyclopentyl]hept-5-enoate in N2B27 medium supplemented with 10 ng/ml of LIF, 10 ng/ml of BMP-4 and 200 ng/ml of GDF6 (see Qi-Long Ying, et al., Cell, Vol. 115, 281-292, Oct. 31, 2003).

The purpose of culturing stem cells may be growth, expansion or maintenance of the stem cells without differentiation of the stem cells.

Using the above-described supplement or medium, stem cells (e.g. embryonic stem cells) can be cultured with or without using feeder cells. Thus, in one embodiment, a method for culturing stem cells (e.g. embryonic stem cells) comprising plating the stem cells in the above-described medium containing the fatty acid derivatives provided by the present invention on a culture container is provided. When stem cells ate cultured without feeder cells, a culture container may be coated with an extracellular matrix. Examples of the extracellular matrix include the extracellular matrix component secreted by a cell (Matrigel® matrix etc.), other component which is extracellularly secreted and enhances cell adhesion, living body (including a human body)-derived collagen, laminin, fibronectin, vitronectin, hyaluronic acid and an artificial synthetic substance of these proteins or polysaccharides (including degradation product and fragment), living body (including a human body)-derived serum, plasma and products separated or purified therefrom. A culture container may be coated with the extracellular matrix by the conventional method.

According to the present invention, the cells may be contacted concomitantly with the compound of the present invention and the one or more stem cell modulators, or the cells may be contacted sequentially.

The present invention may further comprise administering to the subject with one or more stem cell modulators in order to suppress tumorigenicity of the stem cells.

The method of the present invention can be used to stimulate the ex vivo expansion and/or differentiation of stem cells and thereby provide a population of cells suitable for transplantation or administration to a subject in need thereof. Ex vivo expansion of stem cells has therapeutic indications for treating numerous disease conditions.

Sequential methods of stem cells are also contemplated. For example, a stem cell population may be expanded ex vivo by contacting the cells, directly or indirectly, with a fatty acid derivative.

For in vivo and ex vivo methods, the stem cells can be autologous, allogeneic or xenogeneic.

The term “treating” or “treatment” used herein includes prophylactic and therapeutic treatment, and any means of control such as prevention, care, relief of the condition, attenuation of the condition, arrest of progression, etc.

The term “tumorigicity” used herein, includes the ability, tendency or capability to cause, produce or develop tumors. The tumor may be benign (not cancerous), potentially malignant, pre-malignant (pre-cancerous), or malignant (cancerous). The examples of the benign, potentially malignant or pre-malignant tumor used herein include, but not limited to, adenoma, polyp and teratoma.

The term “stem cell” used herein, includes stem cells of human origin or stem cells of non-human mammalian origin and refers to a cell that is capable of differentiating into one or more differentiated cell types. The stem cells may be pluripotent stem cells having the capacity to develop into any cell type, or they may be multipotent stem cells having the capacity to differentiate into several different, final differentiated cell types and derived from a particular tissue or organ, for example, from blood, nerve, skeletal muscle, cardiac muscle, bone marrow, skin, gut, bone, kidney, liver, pancreas, thymus, and the like. Pluripotent stem cells are usually embryonic stem cells in origin and induced pluripotent stem cells and multiipotent stem cells include somatic stem cells such as mesenchymal stem cells, bone marrow stem cells, adipose derived stem cells, hemopoietic stem cells, epidermal stem cells, neuronal stem cells and somatic cells produced by somatic cell transfer. According to the instant invention, stem cells may preferably be neuronal stem cells, epidermal stem cells and mesenchymal stem cells.

The term “progenitor cell” as used herein, refers to a cell that is committed to a particular cell lineage and which gives rise to a particular limited range of differentiated cell types by a series of cell divisions. An example of a progenitor cell would be amyoblast, which is capable of differentiation to only one type of cell, but is itself not fully mature or fully differentiated.

Embryonic stem cells (ESCs) and induced pluripotent stem cells (iPSCs) have the ability to duplicate indefinitely while maintaining pluripotency and to differentiate into cell types of all three embryonic germ layers. These two properties of ESCs and iPSCs make them potentially suitable for tissue engineering and cell replacement therapy for many different diseases. However, one of the critical obstacles in the clinical application of ESCs or iPSCs is the risk of teratoma formation.

Since the fatty acid derivatives of the present invention are useful for suppressing tumorigenicity of stem cells, a high benefit could be expected especially for the clinical application of embryonic and induced pluripotent stem cells.

The pharmaceutical composition of the present invention may contain a single active ingredient or a combination of two or more active ingredients, as far as they are not contrary to the objects of the present invention.

In a combination of plural active ingredients, their respective contents may be suitably increased or decreased in consideration of their therapeutic effects and safety.

The term “combination” used herein means two or more active ingredient are administered to a patient simultaneously in the form of a single entity or dosage, or are both administered to a patient as separate entities either simultaneously or sequentially with no specific time limits, wherein such administration provides therapeutically effective levels of the two components in the body, preferably at the same time.

The present invention will be described in detail with reference to the following example, which, however, is not intended to limit the scope of the present invention.

### Example 1

ESGRO COMPLETE™ C57/BL6 mouse embryonic stem cells (MES, Millipore # SF-CMTI-2) were grown in 0.1% gelatin coated T75 culture flasks using ESGRO complete serum free clonal grade medium (Millipore # SF001-B) and ESGRO complete supplement (Millipore # SF012-250, containing selective GSK3β inhibitor). Cells were treated for 96 hrs with either 1 micromolar of Compound-1 ((−)-7-[(2R,4aR,5R,7aR)-2-(1,1-difluoropentyl)-2-hydroxy-6-oxooctahydrocyclopenta[b]pyran-5-yl]heptanoic acid), Compound-2 ((+)-isopropyl (Z)-7-[(1R,2R,3R,5S)-3,5-dihydroxy-2-(3-oxodecyl)cyclopentyl]hept-5-enoate) or vehicle (0.1% DMSO) until they reached 60% confluence. Drugs and media were changed daily. Cells were incubated in the presence of freshly added drug for 96 hrs, and then tested for mycoplasma over the next 24 hrs. Therefore, the cells were exposed to vehicle or Compound-1 or -2 for a total of 120 hrs. The cells were harvested and mixed with 30% matrigel. The mixture was injected to Fox Chase SCID-beige mice at 1×10⁶ cells/site Tumors were collected after 3 weeks of post injection from kidney and testis. Tumor volumes were measured before and after harvesting from the mice. Tumor weight was also measured after harvesting from mice.

Both the volume and weight of the tumor tissues in kidneys and testes were reduced by treatment with Compound-1 or Compound-2. The results suggest Compound-1 and Compound-2 greatly reduce the tumorigenic potential of mouse ES cells.

TABLE 1
Volume of kidney capsule tumor

| | | Volume of kidney | Volume of kidney |
|---|---|---|---|
| | | capsule tumor, | capsule tumor, |
| | | (within mice) | (after harvesting) |
| | n | mean (cm³) | mean (cm³) |
| Control(water) | 3 | 10.44 | 5.41 |
| Vehicle(DMSO) | 3 | 23.21 | 17.06 |
| Compound-1 | 3 | 5.6 | 2.10 |
| Compound-2 | 3 | 9.12 | 5.44 |

TABLE 2
Volume of testis tumor

| | | Volume of testis | Volume of testis |
|---|---|---|---|
| | | tumor, | tumor, |
| | | (within mice) | (after harvesting) |
| | n | mean (cm³) | mean (cm³) |
| Control(water) | 3 | 21.60 | 11.57 |
| Vehicle(DMSO) | 3 | 12.84 | 8.35 |
| Compound-1 | 3 | 10.42 | 3.70 |
| Compound-2 | 3 | 5.66 | 2.68 |

TABLE 3
Weight of kidney capsule tumor and testis tumor

| | | Weight of kidney | Weight of testis |
|---|---|---|---|
| | | capsule tumor, | tumor, |
| | n | mean (g) | mean (g) |
| Control(water) | 3 | 4.4 | 3.10 |
| Vehicle(DMSO) | 3 | 5.83 | 4.70 |
| Compound-1 | 3 | 1.40 | 1.87 |
| Compound-2 | 3 | 2.13 | 1.33 |

1. A method for suppressing tumorigenicity of stem cells of a mammalian subject, which comprises contacting said stem cell with an effective amount of the fatty acid derivative represented by the formula (I): 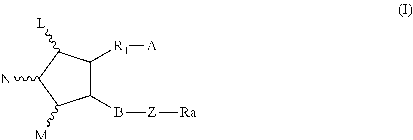
wherein L, M and N are hydrogen, hydroxy, halogen, lower alkyl, hydroxy(lower)alkyl, lower alkanoyloxy or oxo, wherein the five-membered ring may have at least one double bond;
A is —CH₃, or —CH₂OH, —COCH₂OH, —COOH or a functional derivative thereof;
B is single bond, —CH₂—CH₂—, —CH═CH—, —CH₂—CH₂—CH₂—, —CH═CH—CH₂—, —CH₂—CH═CH—, —C≡C—CH₂— or —CH₂—C≡C—;
Z is 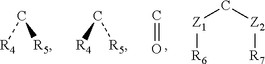
or single bond
wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;
R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

2. The method as described in claim 1, wherein Z is C═O.

3. The method as described in claim 1, wherein B is —CH₂—CH₂—.

4. The method as described in claim 1, wherein B is —CH₂—CH₂— and Z is C═O.

5. The method as described in claim 1, wherein L is hydroxy or oxo, M is hydrogen or hydroxy, N is hydrogen, B is —CH₂—CH₂— and Z is C═O.

6. The method as described in claim 1, wherein the fatty acid derivative is (−)-7-[(2R,4aR,5R,7aR)-2-(1,1-difluoropentyl)-2-hydroxy-6-oxooctahydrocyclopenta[b]pyran-5-yl]heptanoic acid, (+)-isopropyl (Z)-7-[(1R,2R,3R,5S)-3,5-dihydroxy-2-(3-oxodecyl)cyclopentyl]hept-5-enoate, its tautomeric isomers thereof or its functional derivative thereof.

7. The method as described in claim 1, wherein said fatty acid derivative is contacted with the stem cells in vitro or ex vivo.

8. A method for suppressing tumorigenicity of stem cells in a mammalian subject, which comprises administering to the subject in need thereof an effective amount of a fatty acid derivative described in claim 1.

9. The method as described in claim 1, wherein said stem cell is embryonic or induced pluripotent stem cell.

10. The method as described in claim 1, wherein said tumorigenicity is teratoma formation.

11. The method as described in claim 8, wherein said stem cell is embryonic or induced pluripotent stem cell.

12. The method as described in claim 8, wherein said tumorigenicity is teratoma formation.

13. A supplement for culturing stem cells comprising the fatty acid derivative described in claim 1.

14. A medium for culturing stem cells comprising the fatty acid derivative described in claim 1.
